(19) United States
(12) Patent Application Publication
(10) Pub. No.: US 2009/0270401 A1
(43) Pub. Date: Oct. 29, 2009

(54) USE OF A DIHYDROIMIDAZOPYRAZOINE DERIVATIVE FOR TREATING OR PREVENTING PAIN

(75) Inventors: Michel Auguet, Palaiseau (FR); Christine Favre, Saint Maurice Montcouronne (FR); Gregoire Prevost, Antony (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Sceintifiques (S.C.R.A.S.), Paris (FR)

(21) Appl. No.: 11/722,072

(22) PCT Filed: Dec. 16, 2005
(86) PCT No.: PCT/FR2005/003160

§ 371 Date: Jun. 18, 2007

(30) Foreign Application Priority Data
Dec. 17, 2004 (FR) 0413453

(51) Int. Cl. A61K 31/4985; A61P 25/00

(52) U.S. Cl. 514/249

(57) ABSTRACT

The invention relates to the use of (1R)-1-[({(2R)-2-amino-3-[(8S)-8-(cyclohexylmethyl)-2-phenyl-5,6-dihydroimidazo[1,2-a]pyrazine-7(8H)-yl]-3-oxopropyl}dithio)methyl]-2-[(8S)-8-(cyclohexylmethyl)-2-phenyl-5,6-dihydroimidazo[1,2-a]pyrazine-7(8H)-yl]-2-oxoethylamine or an pharmaceutically acceptable salt thereof for preparing a drug for preventing or treating pain.

This invention relates to the use of a dihydroimidazopyrazine derivative, namely (1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo8 1,2-a]pyrazin-7(8H)-yl]-2-oxoethylamine or one of its pharmaceutically acceptable salts for preparing a medicament intended for the prevention or treatment of pain.

Numerous compounds exist which have an analgesic effect, the best known of which is morphine.

Now, certain patients treated with these compounds can show a “tolerance” and even a resistance to the product administered, which is manifested by a reduction in the effectiveness of the compound. In this case it is necessary to increase the doses administered, which can cause side effects.

In order to respond to the requirements of manufacturers it has become necessary to find new compounds for preparing a medicament intended for the prevention or treatment of pain.

Also, the problem which the invention proposes to solve is to provide a novel means suitable for preparing a medicament intended for the prevention or treatment of pain.

Unexpectedly, the inventors have shown that it is possible to use (1R)-1-[({(2R)-2-)-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo [1,2-a]pyrazin-7(8H)-yl]-2-oxoethylamine or one of its pharmaceutically acceptable salts for preparing a medicament intended for the prevention or treatment of pain.

To this end, the present invention proposes the use of (1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine or one of its pharmaceutically acceptable salts for preparing a medicament intended for the prevention and treatment of pain.

The invention has decisive advantages, in particular the use of the compound according to the invention causes little or no sedative effects.

Moreover, advantageously, as the compound according to the invention is a heterodimeric G-protein inhibitor, its use according to the invention allows the effects of multiples mediators involved in pain to be countered.

Other advantages and characteristics of the invention will become clearly apparent on reading the description and the following examples which are given purely by way of illustration and are in no way limitative.

The invention relates to the use of (1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine or one of its pharmaceutically acceptable salts for preparing a medicament intended for the prevention or treatment of pain.

In order to facilitate reading this document, (1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine is hereafter called compound (I).

Preferably, the use according to the invention makes it possible to prevent or treat pain caused by a neuropathy and/or an inflammation.

By the term “pain” is meant in the context of this invention “any unpleasant emotional and sensory experience associated with present or potential tissue damage or described in such terms by the patient” (definition according to the International Association for the Study of Pain (IASP)). Hereafter in this Application, the term pain is used independently to designate the pain or the nociception.

By neuropathy, in the context of the invention, is meant an impairment of function or pathological changes affecting the peripheral nervous system.

More particularly, the pain treated according to the invention is caused by an anti-cancer treatment, such as for example a treatment by chemotherapy or radiotherapy.

Also more particularly, the use according to the invention makes it possible to prevent or treat pain caused by a cancer, and in particular pain associated with metastases, such as for example pain associated with cancers of the prostate, breast, ovaries, pancreas, colon, lung, or skin.

Finally, the use according to the invention makes it possible to prevent or treat pain associated with, among others, inflammation, chronic or otherwise, chronic diseases other than cancer, radiculopathies, adiposis dolorosa, migraine, chronic diseases, fibromyalgia, algoneurodystrophy or complex regional pain syndrome, cerebral vascular accidents, arthrosis, arthritis, diabetic neuropathies or neuropathies following HIV, sciatica, painful muscular contractions, intoxication, burns, post-herpetic neuralgias, thalamic lesions or multiple sclerosis.

Finally, the use according to the invention makes it possible to prevent or treat pain caused by a dermal lesion or by pre- and/or post-operative pain, for example physical pain: traumas or amputations.

Compound (I) or its salt used according to the invention can be in the form of a solid, for example powders, granules, tablets, gelatin capsules, liposomes or suppositories. Suitable supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

Compound (I) or its salt used according to the invention can also be presented in liquid form; for example, solutions, emulsions, suspensions or syrups. Suitable liquid supports can be, for example, water, organic solvents such as glycerol or glycols; similarly their mixtures, in varying in proportions in water.

Administration of compound (I) or its salt used according to the invention can be carried out by topical, oral (p.o.) or parenteral route, by intravenous (i.v.), intramuscular (i.m.), sub-cutaneous (s.c.) injections, etc.

The dose of a product according to this invention envisaged for the treatment of pain mentioned above varies according to the administration method, the age and body weight of the subject to be treated, as well as the subject's state, and it will be decided definitively by the attending doctor or veterinary surgeon. Such a quantity determined by the attending doctor or veterinary surgeon is here termed “effective therapeutic quantity”.

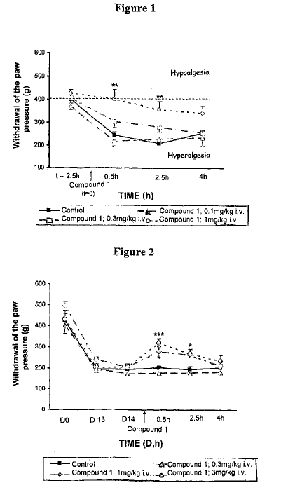

FIG. 1 shows the effect of compound (I) following injection by intravenous route in the carregeenan-induced inflammatory hyperalgesia model.

FIG. 2 shows the effect following injection by intravenous route of compound (I) in the model of neuropathy induced by sciatic nerve lesion.

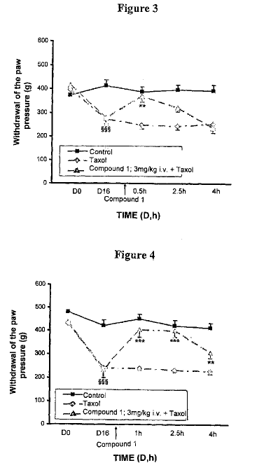

FIGS. 3 and 4 show the effect following injection by intravenous or intra-peritoneal route of compound (I) in the model of neuropathy induced by administration of an anti-cancer agent.

The following examples illustrate the invention without limiting its scope.

### Pharmacological Study of the Products of the Invention

As a reminder, in all the text including the examples hereafter, (1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine is called “Compound (I)”.

Compound (I) corresponds to the following formula:

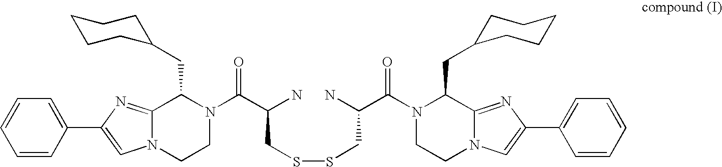

### EXAMPLE 1

### Model of Carrageenin-Induced Inflammatory Hyperalgesia

The activity of compound (I) or its salt according to the invention has been evaluated in vivo on a model of carrageenin-induced inflammatory hyperalgesia in a rat's paw.

Male Sprague Dawley (Charles River) rats weighing approximately 200 g are housed for 5 to 8 days under animal room conditions and fasted on grids for 18 hours before and during the experiment. 4 groups are constituted by at least 6 animals. The products are administered by intravenous route (i.v., 1 ml/kg) 2 hours 30 minutes after the injection of carrageenin. Carrageenin at a concentration of 2 mg/0.1 ml was injected by sub-plantar route in the right rear paw of the rats (0.1 ml was injected per animal). The pain threshold (or nociceptive threshold) was evaluated by measuring the withdrawal of the rat's paw caused by a mechanical stimulus of increasing pressure (initial pressure 210 g/mm²) applied using an analgesy meter (Randall-Selitto test). The measurements were taken just before the injection of carrageenin (t=−2 hours 30 minutes) and then at time 0.5 hours, 2 hours 30 minutes and 4 hours after injection of the products to be tested, which was carried out at t=0.

The effectiveness of the products is evaluated by their ability to reduce significantly the carrageenin-induced hyperalgesia. This effectiveness is statistically determined by a variance analysis test (one route) and/or a Dunnett's test (two routes).

### Effect of Compound (I) on Carraqeenin-Induced Hyperalgesia

The results obtained by using different doses of compound (I) in the model of carrageenin-induced inflammatory hyperalgesia on the rat's paw as described above are shown in FIG. 1.

In FIG. 1, the control indicates the pain threshold tolerated by the rat on its inflamed paw when increasing pressure is applied. At the time of injection of the carrageenin this threshold is between 370 and 430 g/mm² then decreases, indicating that the more the paw is inflamed and painful, the less the rat can bear the application of pressure on it.

The effect of compound (I) indicates that the pain threshold tolerated by the rat on its inflamed paw increases the more compound (I) is administered at significant doses. Starting from the dose of 1 mg/kg (i.v.) of compound (I), the pain threshold following a mechanical stimulus applied to the rats' paws is significantly increased, reaching approximately 400 g/mm² in comparison with 240 g/mm² for the control at time 0.5 hours. This allows the dose-dependant analgesic activity of compound (I) to be demonstrated in this carrageenin-induced hyperalgesia test.

The effective dose (ED₅₀) was determined. By ED₅₀, is meant the dose which reduces by half the pain induced in the model tested.

The ED₅₀ is 0.42 mg/kg at time 0.5 hours in the carrageenin-induced hyperalgesia model.

### EXAMPLE 2

### Model of Neuropathy Induced by Sciatic Nerve Lesion (or “Chronic Constriction Injury”)

The activity of compound (I) or its salt according to the invention was evaluated in vivo on a model of neuropathy induced by chronic constriction injury on the rat's paw.

Male Sprague Dawley (Charles River) rats weighing approximately 200 g are housed for 6 days under animal room conditions. Before the start of the experiment (day zero) the pain threshold (or nociceptive threshold) was evaluated by measuring the withdrawal of the rat's paw caused by a mechanical stimulus (initial pressure 210 g/mm²) applied using an analgesy meter (Randall-Selitto test). This threshold is the pain base threshold. Then, also on day zero, the rats are rendered neuropathic by chronic constriction injury according to the Bennett and Xies (1988) method: 4 ligatures spaced at approximately 2 mm (monocryl suture; 5.0) are carried out on the sciatic nerve of the rat's right paw, above the trifurcation. This operation is carried out under general anaesthetic using isoflurane. On the 13^{th} day after the surgical operation, those rats which are most sensitive to pain are selected from the 32 rats operated on by measuring the withdrawal of the rat's paw caused by a mechanical stimulus of increasing pressure (initial pressure 210 g/mm²) applied using an analgesy meter (Randall-Selitto test). The results allow the most sensitive 28 rats to be selected, 4 groups are constituted by 7 animals. On day 14 after the operation, the pain threshold was re-evaluated. Then compound (I) is administered by intravenous route (i.v.) at 3 different concentrations. The readings are taken at the following times: 0.5 hours 2.5 hours and 4 hours after injection of compound (I). A control group is produced by injection of the vehicle (NaCl solution at 0.9% in g/100 ml) under the same conditions as the product to be tested.

The effectiveness of the products is evaluated by their ability to reduce significantly the pain induced by the chronic constriction injury. This effectiveness is statistically determined by a variance analysis test (one route) and/or a Dunnett's test (two routes).

### Effect of Compound (I) on the Model of Neuropathy Induced by Chronic Constriction Injury:

The results obtained by using different doses of compound (I) in the model of neuropathy induced by chronic constriction injury on the rat's paw as described above are shown in FIG. 2.

In FIG. 2, the control indicates the pain threshold tolerated by the rat on its lesioned paw when increasing pressure is applied. On day zero (before lesion) this threshold was approximately 420 g/mm² then it decreases, reaching approximately 200 g/mm² on day 13. These results indicate that when the paw is lesioned, it becomes painful and it is harder for the rat to bear the application of pressure on it.

The administration of compound (I) indicates that the pain threshold tolerated by the rat on its lesioned paw increases proportionally to the doses of compound (I). Starting from the dose of 3 mg/kg (i.v.) of compound (I), the pain threshold following a mechanical stimulus applied to the rats' paws is significantly increased, to reach approximately 320 g/mm² in comparison with 200 g/mm² for the control at time 0.5 hours. This allows the dose-dependant analgesic activity of compound (I) to be demonstrated in this test of neuropathy induced by chronic constriction injury.

The effective dose (ED₅₀) was determined. By ED₅₀, is meant the dose which reduces by half the pain induced in the model tested.

The ED₅₀ is 2.5 mg/kg at time 0.5 hours in the model of neuropathy induced by chronic constriction injury.

### EXAMPLE 3

### Model of Neuropathy Induced by Administration of an Anti-Cancer Agent.

The activity of compound (I) or its salt according to the invention was evaluated in vivo on a model of neuropathy induced by administration of an anti-cancer agent (paclitaxel (taxol)), on a rat's paw.

Male Sprague Dawley (Charles River) rats weighing approximately 200 g are housed for 6 days under animal room conditions. 3 groups are constituted by at least 10 animals.

The neuropathy is induced by intra-peritoneal injections (i.p.) of 2 mg/kg of paclitaxel (extract of Taxus Yannanensis) on days D0, D2, D4 and D7.

Before the first injection, the rats are numbered and weighed and nociception is evaluated after a mechanical stimulus of increasing pressure: induction of an initial pressure (210 g/m²) on the rats' two rear paws carried out using an analgesy meter according to the Randall and Selitto method. These measurements allow the base values to be defined before development of the neuropathy (D0).

The decrease in the nociceptive threshold corresponding to the neuropathic impairment is at a maximum between the 16^{th} and the 24^{th} day after the first injection of paclitaxel. The nociception threshold of the rats' two rear paws is decreased similarly, allowing the values to be averaged in order to facilitate the calculations. The neuropathy protection studies are thus carried out between the 16^{th} and the 24^{th} day.

On the day of the experiment, the rats are weighed, nociception is measured and the animals which have not developed the neuropathy on this day (reduction of nociception in comparison with the readings on D0) are excluded from the study. The product being tested is administered and nociception is measured at different times after its administration.

### Effect of Compound (I) on Taxol-Induced Neuropathy

The results obtained by using different doses of compound (I) in the taxol-induced neuropathy model are shown in FIG. 3.

In FIG. 3, the control indicates the pain threshold tolerated by the rat when increasing pressure is applied on its paws. On day zero this threshold is approximately 400 g/mm², then decreases reaching approximately 270 g/mm² on day 16. These results indicate that after i.p. injection of paclitaxel, the sensitivity of rats' paws is increased following the application of pressure on them.

The administration of compound (I) indicates that the pain threshold tolerated by the rat on its paws after induction of the neuropathy by paclitaxel increases with compound (I) at the dose of 3 mg/kg (i.v.). The pain threshold following a mechanical stimulus applied on the rats' paws is significantly increased, to reach approximately 370 g/mm² in comparison with 250 g/mm² for the paclitaxel alone at time 0.5 hours. This allows the analgesic activity of compound (I) to be demonstrated in this test of neuropathy induced by chronic systemic injection of paclitaxel.

In FIG. 4, the control indicates the pain threshold tolerated by the rat when increasing pressure is applied on its paws. On day zero this threshold is approximately 450 g/mm², then decreases reaching approximately 235 g/mm² on day 16. These results indicate that after i.p. injection of paclitaxel, the sensitivity of rats' paws is increased following the application of pressure on them.

The administration of compound (I) indicates that the pain threshold tolerated by the rat on its paws after induction of the neuropathy by paclitaxel increases with compound (I) at the dose of 100 mg/kg (p.o.). The pain threshold following a mechanical stimulus applied to the rats' paws is significantly Increased, reaching approximately 400 g/mm² in comparison with 240 g/mm² for paclitaxel alone at time 1 hour. This allows the analgesic activity of compound (I) to be demonstrated in this test of neuropathy induced by chronic systemic injection of paclitaxel.

1. A method of preventing or treating pain comprising administering a medicament comprising (1R)-1-[({(2R)-2-amino-3-[(8S)-8-(cyclohexylmethyl)-2-phenyl-5,6-dihydroimidazo[1,2-α]pyrazin-7(8H)-yl]-3-oxopropyl}dithio)methyl]-2-[(8S)-8-(cyclohexylmethyl)-2-phenyl-5,6-dihydroimidazo[1,2-α]pyrazin-7(8H)-yl]-2-oxoethylamine or a pharmaceutically acceptable salts thereof.

2. The method of claim 1, wherein the pain is caused by a neuropathy and/or an inflammation.

3. The method of claim 1, wherein the pain is caused by an anti-cancer treatment.

4. The method of claim 3, wherein the anti-cancer treatment is a treatment by chemotherapy or radiotherapy.

5. The method of claim 1, wherein the pain is caused by a cancer.

6. The method of claim 1, characterized in that wherein the pain is associated with an inflammation, chronic or otherwise, chronic diseases other than cancer, radiculopathies, adiposis dolorosa, migraine, chronic diseases, fibromyalgia, algoneurodystrophy or complex regional pain syndrome, cerebrovascular accidents, arthrosis, arthritis, diabetic neuropathies or following HIV, sciatica, painful muscular contractions, intoxication, burns, post- herpetic neuralgias, thalamic lesions or multiple sclerosis.

7. The method of claim 1, wherein the pain is caused by a dermal lesion or by pre- and/or post-operative pain.

8. The method of claim 5, wherein the pain caused by cancer is pain associated with cancers of the prostate, breast, ovaries, pancreas, colon, lung or skin.
